SEQUENCE LISTING (4 sequences)

SEQ ID NO: 1 - DNA, 20 bp, Artificial Sequence
  COX-2 primer (F)
aagacttgccaggctgaact

SEQ ID NO: 2 - DNA, 20 bp, Artificial Sequence
  COX-2 primer (R)
cttctgcagtccaggttcaa

SEQ ID NO: 3 - DNA, 27 bp, Artificial Sequence
  GAPDH primer (F)
tgaaggtcggtgtgaacggattttggc

SEQ ID NO: 4 - DNA, 25 bp, Artificial Sequence
  GAPDH primer (R)
tggttcacacccatcacaaacatgg

(19) United States
(12) Patent Application Publication
(10) Pub. No.: US 2023/0042326 A1
(43) Pub. Date: Feb. 9, 2023

(54) SKIN SOOTHING COMPOSITION INCLUDING EXOSOMES DERIVED FROM NATURAL EXTRACT

(71) Applicant: MEDICAL O CO., LTD., Seoul (KR)

(72) Inventors: Dong Hoon JANG, Seoul (KR); In Moo BYUN, Gyeonggi-do (KR); Jee Yong KIM, Gyeonggi-do (KR)

(73) Assignee: MEDICAL O CO., LTD., Seoul (KR)

(21) Appl. No.: 17/382,855

(22) Filed: Jul. 22, 2021

(51) Int. Cl. A61K 35/12; A61K 8/98; A61Q 19/08; A61K 8/02; A61P 17/00; A61P 29/00

(52) U.S. Cl. CPC A61K 35/12; A61K 8/981; A61Q 19/08; A61K 8/0212; A61P 17/00; A61P 29/00

(57) ABSTRACT

Disclosed is a cosmetic composition for skin soothing. The cosmetic composition includes, as active ingredients, exosomes derived from naturally occurring deer antler velvet. The cosmetic composition can effectively soothe the skin whose condition has been abnormally altered (such as atopic dermatitis, inflammation, erythema, oxidation, cytotoxic agents in the skin, loss of moisture, melasma, itching, roughness or wrinkles) by various causes.

### BACKGROUND OF THE INVENTION

### 1. Field of the Invention

The present invention relates to a skin soothing composition including exosomes derived from a natural extract.

### 2. Description of the Related Art

All cells are able to release extracellular vesicles (EVs). This extracellular vesicle release is an evolutionarily conserved phenomenon from bacteria to humans and plants. Extracellular vesicles enable intercellular exchange of materials such as proteins, lipids, and genetic materials and function as mediators to transmit physiological/pathological signals. Extracellular vesicles are broadly classified into exosomes and microvesicles. Exosomes vary in size depending on their biological origin (for example, 30-200 nm for human-derived exosomes). Intraluminal vesicles are formed by inward budding of endosomal membranes during maturation of multivesicular endosomes. Exosomes are released when multivesicular endosomes fuse with the cell surface. Microvesicles are vesicles with a size of 50-1000 nm that are formed by outward budding of plasma membranes and are released extracellularly. Cells produce different extracellular vesicles depending on their physiological state and release extracellular vesicles with specific lipid/protein/nucleic acid compositions (Jaewook Lee (2019). Studies of the cell biology of extracellular vesicles. BRIC View 2019-R03).

Exosomes contain specific genetic materials and bioactive factors depending on the nature and state of cells from which they are derived. That is, exosomes contain various types of proteins, genetic materials (e.g., DNAs, mRNAs, and miRNAs), lipids, and other substances derived from their cellular origin. Such exosomes have different characteristics from proteins and genetic materials present in biofluids (e.g., blood, serum, and lymph). Specifically, exosomes are spherical particles surrounded by lipid bilayers and carry various cell-derived proteins on their surface and cell-derived genetic materials therein. Particularly, genetic materials released from cells tend to degrade due to the presence of excess ribonuclease (RNase) in serum. In contrast, genetic materials present in exosomes are protected from RNase. This stability allows genetic materials in exosomes to possess different activities and therapeutic effects from cells or cell cultures.

Deer antler velvet refers to a medicinal material obtained by harvesting and processing the unossified antlers of young Formosan and red stags. When deer antler velvet is provided to a weak child who is growing slower or suffering from cartilage disease, it helps not only promote his/her growth and strengthen his/her skeleton but also develops his/her intelligence and effectively increases the digestion and absorption capacity of his/her stomach and intestines. Deer antler velvet is known to cause sexual arousal and increase sexual desire due to the presence of large amounts of hormones, particularly estrogenic hormones (Encyclopedia of Korean National Culture, The academy of Korean studies).

Dermatitis refers to an inflammation of the skin, including eczema. Dermatitis is usually classified into atopic dermatitis, contact dermatitis, and seborrheic dermatitis (quoted from Doosan Encyclopedia).

(1) Atopic Dermatitis

Atopic dermatitis is an eczema-like skin lesion that occurs in people with atopic constitution. It also called endogenous eczema or Besnier's prurigo. Atopic dermatitis tends to be hereditary but its cause is unknown. Unlike normal eczema or dermatitis, atopic dermatitis has specific symptoms and progresses. Atopic dermatitis accounts for 70-80% of all pediatric patients with eczema. Symptoms of atopic dermatitis vary with age. The course of atopic dermatitis is usually divided into three phases: \textcircled{1} Infancy (around 2 months to 3 years of age)—Redness, exudation, and desquamation appear on the face, especially the cheeks. Another symptom is extreme itchiness. When atopic dermatitis in infancy worsens, the same symptoms and scabies also appear on the head, the skin becomes red and desquamates all over the body, and the entire skin becomes rough and bluish-white. Atopic dermatitis starts in around 2- to 3-month-old infants and is cured well until 1 year of age but is often recurrent. In general, atopic dermatitis tends to get worse in winter; \textcircled{2} Childhood (around 4 to 10 years of year)—Papules and prurigoes appear on the extremities (especially the flexion of the elbow and knee joints) of children at the age of around 4-5 years and fuse to form lichens; and \textcircled{3} Adolescence (beyond 12 years of age)—Lichens appear on the face, chest, and back of the neck as well as the extremities. Atopic dermatitis is also accompanied by complications such as pediatric asthma and other family members are infected with asthma or atopic dermatitis. Since atopic dermatitis is a disease that is long-lasting and is difficult to treat, it is important to treat patients with atopic dermatitis with a relaxed mind and with patience. The symptoms of atopic dermatitis become less severe with age. The application of ointments (e.g., antihistamine ointments and vitamin A/vitamin D ointments) along with the administration of antipruritic drugs is recommended for severe symptoms of atopic dermatitis.

(2) Contact Dermatitis:

Contact dermatitis is an inflammation of the skin that occurs upon contact with foreign materials. It is characterized by skin itching, so-called skin sore. Contact dermatitis has the same symptoms as acute eczema but is different from eczema in that it occurs in response to specific foreign materials. Materials tending to cause contact dermatitis include plants such as poison oaks, poison sumacs, fig trees, and maidenhair tree, paints, synthetic resin products, leather products, rubber products, chromium plated products, pharmaceuticals, cosmetics, and synthetic fiber underwear. Contact dermatitis is prevented by avoiding contact with causative agents and stopping facial makeup and scratching itchy skin. For mild symptoms, boric acid and zinc oxide ointments are spread or antihistamines, adrenocortical hormones, vitamin B2, vitamin B6, etc. for internal use are administered. If the symptoms of contact dermatitis are not treated in 2-3 days or become severe, patients should it is recommended to see a dermatologist.

(3) Seborrheic Dermatitis:

Seborrheic dermatitis is a dermatitis that occurs predominantly in seborrheic regions such as the head, forehead, and armpits. It is also called seborrheic eczema. The most common symptoms of seborrheic dermatitis are erythema and scaling scalp (dandruff). Seborrheic dermatitis frequently occurs at the age of 20s to 40s. Unlike typical eczema, seborrheic dermatitis is caused by constitution or abnormal secretion of sebum. The mildest symptoms are dandruff and psoriasis. Seborrheic dermatitis is sensitive to sunlight or heat, often worsens in spring and autumn, and even recurs easily. Seborrheic dermatitis is prevented by avoiding fat food and stopping scratching and irritating the affected areas such as the head by scratching with nails. Seborrheic dermatitis is treated by administration of vitamin B2, vitamin B6, nicotinamide, etc., similarly to eczema.

For the treatment of seborrheic dermatitis, it is common to prescribe steroids (e.g., topical corticosteroids) and antihistamines. However, long-term use of topical corticosteroids causes various dermatologic side effects such as anetoderma, vasodilation, depigmentation, and formation of striae distensae. Further, resistance and hypersensitivity to antihistamines are problematic.

Until now, there have been no reports that deer antler velvet-derived exosomes normalize or soothe skin disorders including dermatitis. Thus, there is an urgent need to develop skin soothing cosmetics derived from natural products.

The description of the Background Art is merely provided for better understanding the background of the invention and should not be taken as corresponding to the prior art already known to those skilled in the art.

### SUMMARY OF THE INVENTION

The present inventors have made an effort to find naturally occurring materials that can soothe the skin whose condition has been abnormally altered by various causes such as genetic and environmental factors and aging, and as a result, found that exosomes derived from deer antler velvet are very effective in soothing the skin. The present invention has been accomplished based on this finding.

Accordingly, one object of the present invention is to provide a skin soothing composition including deer antler velvet-derived exosomes as active ingredients.

A further object of the present invention is to provide a method for preparing a skin soothing composition including deer antler velvet-derived exosomes as active ingredients.

Another object of the present invention is to provide a method for soothing skin in a subject in need thereof, comprising administering to the subject a composition comprising deer antler velvet-derived exosomes as active ingredients.

Another object of the present invention is to provide a method for ameliorating or treating dermatitis, atopic dermatitis or wrinkle in a subject in need thereof, comprising administering to the subject a composition comprising deer antler velvet-derived exosomes as active ingredients. Other objects and advantages of the invention become more apparent from the following detailed description, claims, and drawings.

One aspect of the present invention provides a skin soothing composition including deer antler velvet-derived exosomes as active ingredients.

In an attempt to develop natural cosmetics and/or pharmaceuticals, the present inventors have extracted ingredients from deer antler velvet by various techniques and observed and compared their effects for a long period of time. As a result, the present inventors have found that the clinical efficacies of the deer antler velvet extracts are significantly different depending on the extraction techniques. It has surprisingly been found that exosomes derived from deer antler velvet are very effective in soothing the skin.

As used herein, the term “exosomes” refers to vesicular structures composed of lipid bilayers that are naturally released from various types of cells to the extracellular environment while harboring specific molecules present in the cells, such as proteins, nucleic acids (e.g., DNAs, mRNAs, and miRNAs), lipids, and carbohydrates. This term has the same meaning as the term “extracellular vesicles”.

The diameter of exosomes or extracellular vesicles varies in the range of about 30 to about 1,000 nm depending on their animal or cellular origin. The diameter of the deer antler velvet-derived exosomes is preferably 100 to 800 nm, most preferably 120 to 600 nm.

In the Examples section that follows, the deer antler velvet-derived exosomes or extracellular vesicles used in the composition of the present invention were analyzed to have a diameter of about 120 to 600 nm (FIG. 2A).

The exosomes or extracellular vesicles are surrounded by lipid bilayers to protect proteins, nucleic acids, lipids, and carbohydrates from the activity of various enzymes such as ribonuclease (RNase) and protease present in biofluids such as blood, plasma or lymph. Thus, the exosomes or extracellular vesicles may have a completely different composition from that of the blood or lymph, cells present in the blood or lymph or a culture of these cells.

As used herein, the term “skin soothing” refers to a process for soothing the skin whose condition has been abnormally altered (such as atopic dermatitis, inflammation, erythema, oxidation, cytotoxic agents in the skin (e.g., nitric oxide (NO) accumulation), loss of moisture, melasma, itching, roughness or wrinkles) by various causes such as genetic and environmental factors and aging to allow the skin to return to its normal condition.

In the Examples section that follows, the deer antler velvet-derived exosomes or extracellular vesicles used in the composition of the present invention were found to effectively inhibit the expression of thymus and activation-regulated chemokine (TARC), a specific factor for atopic dermatitis, and significantly suppress the production of overexpressed MDC induced by TNF-α and IFN-γ, whereas these effects were hardly observed in a general deer antler velvet extract, demonstrating that the deer antler velvet-derived exosomes or extracellular vesicles are very useful as immunosuppressants (e.g., for atopic dermatitis) (FIGS. 4 and 5).

In the Examples section that follows, it was also found that the deer antler velvet-derived exosomes or extracellular vesicles used in the composition of the present invention suppressed the production of cytotoxic nitric oxide (NO) in all treated groups compared to an LPS-treated control, unlike a general deer-antler velvet extract (FIG. 6).

In the Examples section that follows, a significant inhibitory effect on COX-2 gene expression was observed in groups treated with the deer antler velvet-derived exosomes or extracellular vesicles used in the composition of the present invention compared to in a negative control treated with LPS alone and a deer antler velvet extract, demonstrating that the deer antler velvet-derived exosomes or extracellular vesicles can be used to treat and prevent inflammatory diseases or are effective in skin soothing (FIG. 7).

In the Examples section that follows, the deer antler velvet-derived exosomes or extracellular vesicles used in the composition of the present invention effectively suppressed the production of MMP-1 protein that causes the formation of wrinkles upon skin dryness, environmental changes such as ultraviolet radiation, and aging, demonstrating that the deer antler velvet-derived exosomes or extracellular vesicles can be effectively used to prevent and improve skin wrinkles (FIG. 8).

The composition of the present invention may be used to prepare a cosmetic composition. In this case, the composition of the present invention includes one or more components commonly used in cosmetic compositions, in addition to the deer antler velvet-derived exosomes or extracellular vesicles. Examples of such components include adjuvants such as antioxidants, stabilizers, solubilizing agents, vitamins, pigments and fragrances, and carriers. The composition of the present invention optionally includes one or more skin soothing agents commonly used in the art, in addition to the deer antler velvet-derived exosomes or extracellular vesicles.

Examples of the carriers include, but are not limited to, purified water, monohydric alcohols (e.g., ethanol and propyl alcohol), polyhydric alcohols (e.g., glycerol, 1,3-butyrene glycol, and propylene glycol), higher fatty acids (e.g., palmitic acid and linolenic acid), and oils and fats (e.g., wheat germ oil, camellia oil, jojoba oil, olive oil, squalene, sunflower oil, macadamia nut oil, avocado oil, hydrogenated soybean lecithin, and fatty acid glycerides). The composition of the present invention may optionally further include a surfactant, a disinfectant, an antioxidant, an ultraviolet light absorber, an anti-inflammatory agent, and/or a refreshing agent.

The surfactant may be selected from the group consisting of polyoxyethylene, hydrogenated castor oil, polyoxyethylene, oleyl ether, polyoxyethylene monooleate, polyoxyethylene, glyceryl monostearate, sorbitan monostearate, polyoxyethylene monooleate, sorbitan, sucrose fatty acid ester, hexaglycerin monolaurate, polyoxyethylene reduced lanolin, POE, glyceryl pyroglutamate, isostearic acid diester, N-acetylglutamine, and isostearyl ester.

The disinfectant may be selected from the group consisting of hinokitiol, triclosan, chlorhexidine gluconate, phenoxyethanol, resorcin, isopropylmethylphenol, azulene, salicylic acid, and zinc pyrithione.

The antioxidant may be selected from the group consisting of butylated hydroxyanisole, gallic acid, propyl gallate, and erythorbic acid.

The ultraviolet light absorber may be selected from the group consisting of benzophenones such as dihydroxybenzophenone, melanin, ethyl para-aminobenzoate, 2-ethylhexyl para-dimethylaminobenzoate, cinoxate, 2-ethylhexyl para-methoxycinnamate, 2-(2-hydroxy-5-methylphenyl)benzotriazole, urocanic acid, and fine metal oxide particles.

The anti-inflammatory agent may be dipotassium glycyrrhetinate or allantoin. The refreshing agent may be capsicum tincture or 1-menthol.

The composition is prepared into any formulation in which the deer antler velvet-derived exosomes or extracellular vesicles as active ingredients can be blended. The composition may be prepared into cosmetic formulations. Examples of such cosmetic formulations include, but are not limited thereto, powders, gels, creams, essences, lotions, sol-gels, emulsions, oils, waxes, sprays, and mists. The deer antler velvet-derived exosomes or extracellular vesicle or the composition including the same may be used to prepare mask packs.

A further aspect of the present invention provides a food composition or pharmaceutical composition including the deer antler velvet-derived exosomes.

Another aspect of the present invention provides a method for soothing skin in a subject in need thereof, comprising administering to the subject a composition comprising deer antler velvet-derived exosomes as active ingredients.

Another aspect of the present invention provides a method for ameliorating or treating dermatitis, atopic dermatitis or wrinkle in a subject in need thereof, comprising administering to the subject a composition comprising deer antler velvet-derived exosomes as active ingredients.

In addition to the deer antler velvet-derived exosomes or extracellular vesicles as active ingredients, the food composition of the present invention includes one or more ingredients that are usually added for food production. For example, the food composition of the present invention may include one or more ingredients selected from proteins, carbohydrates, fats, nutrients, seasoning agents, and flavoring agents. Examples of the carbohydrates include: saccharides such as monosaccharides (e.g., glucose and fructose), disaccharides (e.g., maltose, sucrose, and oligosaccharides), and polysaccharides (e.g., dextrin and cyclodextrin); and sugar alcohols such as xylitol, sorbitol, and erythritol. The flavoring agents may be natural flavoring agents such as thaumartin and stevia extracts (e.g., rebaudioside A and glycyrrhizin) and synthetic flavoring agents (e.g., saccharin and aspartame).

For example, the food composition of the present invention may be prepared into a drink. In this case, the food composition of the present invention may further include citric acid, high fructose corn syrup, sugar, glucose, acetic acid, malic acid, fruit juice, eucommia extract, jujube extract, licorice extract, etc.

The food composition of the present invention is very effective in soothing the skin. Moreover, the composition of the present invention is very safe to humans because the active ingredients are natural products whose biosafety has already been proven in humans.

According to a preferred embodiment of the present invention, the pharmaceutical composition is used to prevent or treat skin inflammatory diseases, atopic dermatitis or skin wrinkles.

The pharmaceutical composition of the present invention may include one or more pharmaceutically acceptable carriers that are commonly used for formulation. Examples of the pharmaceutically acceptable carriers include, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil. The pharmaceutical composition of the present invention may further include one or more additives selected from the group consisting of lubricating agents, wetting agents, sweetening agents, flavoring agents, emulsifying agents, suspending agents, and preservatives. Details of suitable pharmaceutically acceptable carriers and formulations can be found in Remington's Pharmaceutical Sciences (19th ed., 1995).

The pharmaceutical composition of the present invention can be administered orally or parenterally. Examples of suitable parenteral routes of administration include intranasal administration, eye drop administration, intravenous injection, subcutaneous injection, intramuscular injection, intraperitoneal injection, and transdermal administration.

A suitable dose of the pharmaceutical composition according to the present invention may vary depending on factors such as formulation, mode of administration, patient's age, weight, sex, pathological condition, and diet, time of administration, route of administration, excretion rate, and responsiveness. A skilled physician can easily determine and prescribe a dose of the pharmaceutical composition according to the present invention effective for desired treatment or prevention. According to a preferred embodiment of the present invention, the pharmaceutical composition is administered in a daily dose of 0.001 to 100 mg/kg.

The pharmaceutical composition of the present invention can be formulated with one or more pharmaceutically acceptable carriers and/or excipients in accordance with methods that can be easily carried out by those skilled in the art. The pharmaceutical composition can be provided in unit dosage forms or dispensed in multi-dose containers. The formulation may be in the form of a solution, suspension or emulsion in an oil or aqueous medium or may be in the form of an extract, powder, granule, tablet or capsule. The formulation may further include a dispersant or a stabilizer.

The pharmaceutical composition of the present invention may be formulated into skin preparations for external use, aerosols, sprays, eye drops, oral preparations, pharmacopuncture solutions, and injectable preparations.

When the pharmaceutical composition of the present invention is formulated into an injectable preparation, the blending proportion of the deer antler velvet-derived exosomes or extracellular vesicles may also be appropriately selected depending on the type of the injectable preparation and the kind, amount, and form of other ingredients to be blended. The deer antler velvet-derived exosomes or extracellular vesicles are typically present in an amount of 0.01 to 99% by weight, preferably 0.1 to 30% by weight, based on the total weight of the injectable preparation.

In addition to the deer antler velvet-derived exosomes or extracellular vesicles, the injectable preparation may further include one or more other ingredients that do not impair the desired effect of the present invention. The additional ingredients may be appropriately selected from water for injection, NaCl, NaOH, and glucose.

As used herein, the terms “cosmetic composition”, “food composition”, and “pharmaceutical composition” are intended to include applications to animals, including livestock and household pets, as well as to humans.

As used herein, the term “subject”, “patient”, and “individual” are interchangeable and refer to any mammalian subject to which any of the compositions described herein are administered. Non-limiting examples include humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications.

As used herein, the phrase “subject in need thereof” includes subjects, such as mammalian subjects, that would benefit from administration of the composition described herein.

As used herein, the term “therapeutically effective amount” is the amount of reagent or pharmaceutical compound comprising a composition of the present disclosure that is sufficient to a produce a desired therapeutic effect, pharmacologic and/or physiologic effect on a subject in need thereof. A therapeutically effective amount can be a “prophylactically effective amount” as prophylaxis can be considered therapy.

Another aspect of the present invention provides a method for preparing a skin soothing composition including deer antler velvet-derived exosomes as active ingredients, the method including a) washing and powdering deer antler velvet, b) dissolving the powder in an exosome extraction buffer, and c) isolating deer antler velvet-derived exosomes from the solution.

The exosome extraction buffer means a buffer capable of dissolving the exosome powder. The exosome extraction buffer can be selected from exosome isolation kits widely used in the art, including but not limited to, EXO-BB, ExoQuick®-ULTRA, ExoQuick®-TC, Capturem™ Exosome Isolation Kit, Total Exosome Isolation Kit, ExoTrap™ Exosome Isolation Spin Column Kit, and Exo2D™.

As used herein, the term “isolation” is intended to include both positive isolation, where target substances (e.g., exosomes) are selectively obtained in a biological sample (e.g., a solution of a deer antler velvet powder in an exosome extraction buffer), and negative isolation, where impurities other than the target substances are selectively removed. Accordingly, the term “isolating” is synonymous with “obtaining”, “extracting”, and “purifying”. Any process for exosome isolation known in the art may be used without limitation in the present invention. For example, the exosomes may be isolated using the aforementioned commercially available exosome isolation kits. Alternatively, the exosomes may be isolated by suitable techniques based on the inherent physical properties of target materials in heterogeneous samples that are commonly used in the art, including but not limited to, an isolation technique based on a difference in specific gravity between components in a solution (e.g., centrifugation), an isolation technique based on a difference in size between components in a solution (e.g., ultrafiltration and vacuum filtration), and an isolation process based on an affinity for a particular substrate (e.g., affinity chromatography).

The composition prepared by the method of the present invention may be a cosmetic composition, food composition or pharmaceutical composition. The composition significantly inhibits TARC expression and COX-2 gene expression and significantly suppresses the production of overexpressed MDC induced by TNF-α and IFN-γ to effectively suppress inflammations, including atopic dermatitis, unlike general deer antler velvet extracts. In addition, the composition of the present invention suppresses the production of cytotoxic nitric oxide to effectively sooth the skin whose condition has been abnormally altered by various causes such as genetic and environmental factors and aging.

The features and advantages of the present invention are summarized as follows:

(i) The cosmetic composition of the present invention, which includes deer antler velvet-derived exosomes as natural active ingredients, is effective in soothing the skin.

(ii) The composition of the present invention can effectively soothe the skin whose condition has been abnormally altered (such as atopic dermatitis, inflammation, erythema, oxidation, cytotoxic agents in the skin, loss of moisture, melasma, itching, roughness or wrinkles) by various causes.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIGS. 1A, 1B and 1C show the contents of CD9 (FIG. 1A), CD63 (FIG. 1B), and CD81 proteins (FIG. 1C) in deer antler velvet-derived exosomes;

FIGS. 2A and 2B show the diameters of exosomes derived from deer antler velvet (FIG. 2A) and tortoise shell (FIG. 2B) determined by transmission electron microscopy and DLS.

FIG. 3 shows the effect of deer antler velvet-derived exosomes on cytotoxicity.

FIG. 4 shows the anti-atopic efficacy of deer antler velvet-derived exosomes (inhibitory efficacy on the production of overexpressed TARC induced by TNF-α and IFN-γ);

FIG. 5 shows the anti-atopic efficacy of deer antler velvet-derived exosomes (inhibitory efficacy on the production of overexpressed MDC induced by TNF-α and IFN-γ);

FIG. 6 shows the inhibitory efficacy of deer antler velvet-derived exosomes on nitric oxide production;

FIG. 7 shows the anti-inflammatory efficacy of deer antler velvet-derived exosomes; and

FIG. 8 shows the anti-wrinkle efficacy of deer antler velvet-derived exosomes.

### DETAILED DESCRIPTION OF THE INVENTION

The present invention will be more specifically explained with reference to the following examples. It will be evident to those skilled in the art that the scope of the present invention is not limited by these examples according to the gist of the present invention.

### EXAMPLES

### 1. Preparation of Deer Antler Velvet-Derived Exosomes (Experimental Example 1)

10 g of deer antler velvet (Hans Medicine Co., Ltd., Korea) was washed with purified water, mixed, and dried to prepare a mixed sample. The mixed sample was pulverized into a finely divided powder, followed by drying. A 10-fold volume of a buffer (EXO-BB, System Biosciences, Palo Alto, Calif., USA) was added to the dry powder. The dry powder was dissolved by vortexing for 5 min. The solution was placed in a 2 mL collection tube, loaded onto a filter cartridge, and centrifuged at 18,000 G for 2 min. The down layer was transferred to a 1.5 mL microfuge tube. At that time, care was taken not to permit pellets to fall apart from the tube during centrifugation. A 2-fold volume of an exosome isolation buffer was added to the microfuge tube, followed by incubation at 4° C. for 48 h to increase the yield of exosomes. Thereafter, centrifugation was performed at 10,000 G for 60 min. The supernatant was removed from the microfuge tube to isolate deer antler velvet-derived exosomes in the form of pellets. The exosome pellets were redissolved in EXO-BB buffer using a sonicator and stored at 4° C. until use for subsequent experiments.

### 2. Preparation of Deer Antler Velvet Extract (Experimental Example 2)

A deer antler velvet extract was prepared, and its activity was compared with that of the deer antler velvet-derived exosomes. 100 g of deer antler velvet (Hans Medicine Co., Ltd., Korea) was washed with purified water, mixed, and dried to prepare a mixed sample. The mixed sample was pulverized into a finely divided powder, followed by drying. The dry powder was extracted by heating under reflux in 1 L of purified water as a solvent for 12 h, macerated, and filtered through a filter paper having a pore size of 1.2 μm. The filtrate was concentrated and dried under reduced pressure to prepare a deer antler velvet extract in the form of a dry powder.

### 3. Identification of the Deer Antler Velvet-Derived Exosomes

1) Identification Using Exosome Markers

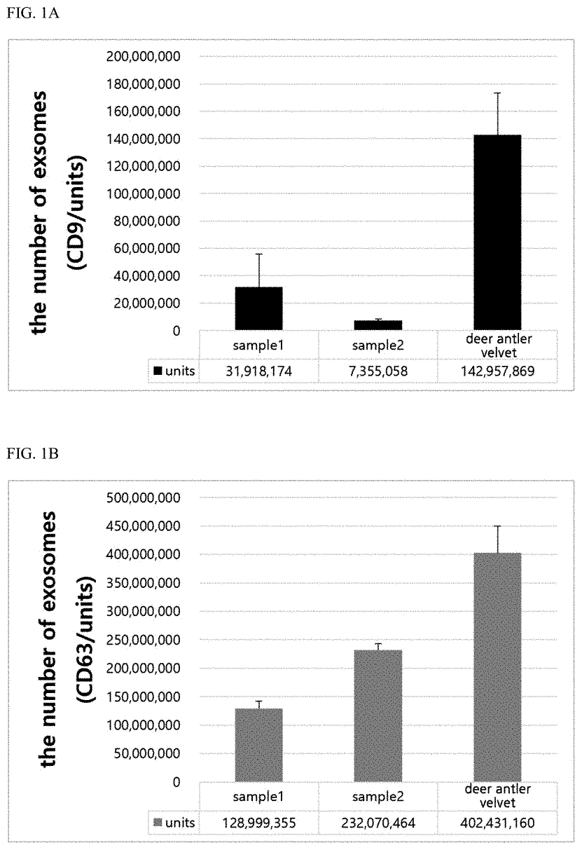

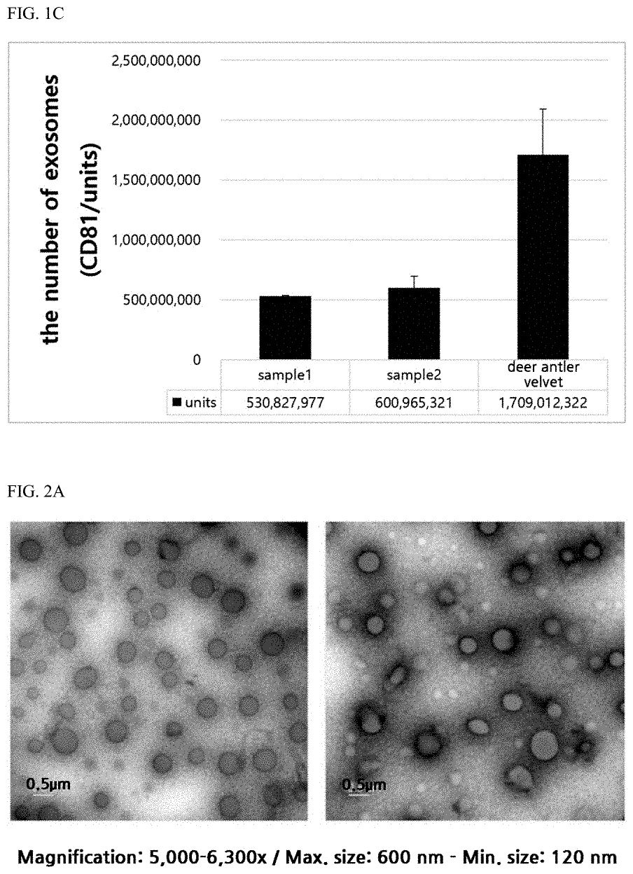

The contents of CD9, CD63, and CD81 proteins in the deer antler velvet-derived exosomes prepared in Experimental Example 1 were analyzed using an enzyme-linked immunosorbent assay (ELISA) kit (System Biosciences, Palo Alto, Calif., USA). CD9, CD63, and CD81 proteins are typical exosome markers. A tortoise shell extract (sample 1) and a musk extract (sample 2) were used as controls for comparative experiments. The tortoise shell extract and the musk extract were prepared in the same manner as in Experimental Example 1, except that tortoise shell (Daon Co., Ltd., Korea) and musk (Seosahyang Co., Ltd., Korea) were used as raw materials, respectively. Tortoise shell and musk have been used as animal-derived medicines in Korea. As a result, the deer antler velvet-derived exosomes prepared in Experimental Example 1 were found to contain significantly larger amounts of exosome markers compared to the tortoise shell and musk extracts (samples 1 and 2), indicating the presence of a much greater number of exosomes in the deer antler velvet than in the animal-derived medicines (FIG. 1A: CD9, FIG. 1B: CD63, and FIG. 1C: CD81).

2) Analysis Using Transmission Electron Microscope

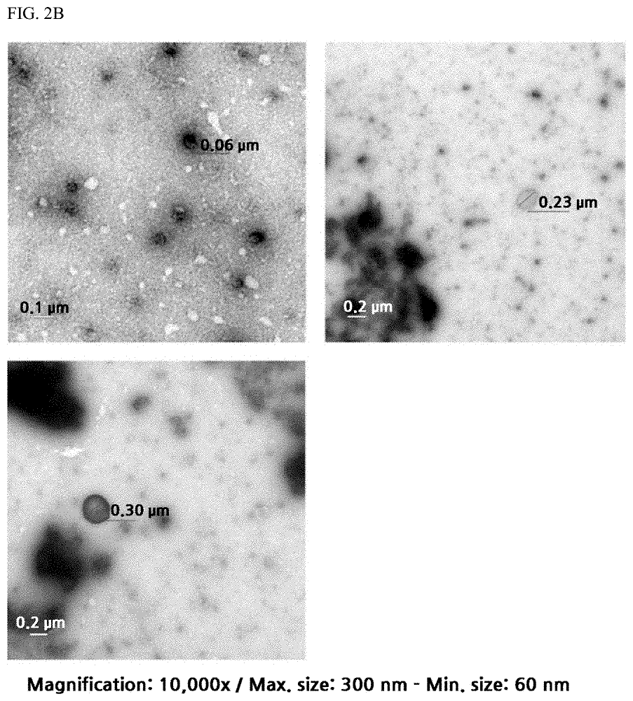

The shapes and sizes of the deer antler velvet-derived exosomes prepared in Experimental Example 1 and the tortoise shell-derived exosomes (sample 1) were analyzed by transmission electron microscopy and dynamic light scattering (DLS). As a result, the deer antler exosomes and the tortoise shell-derived exosomes were substantially spherical in shape and had diameters of ˜60-600 nm (FIG. 2A: deer antler velvet-derived exosomes; FIG. 2B: tortoise shell-derived exosomes).

### 4. Evaluation of Effect of the Deer Antler Velvet-Derived Exosomes on Cytotoxicity

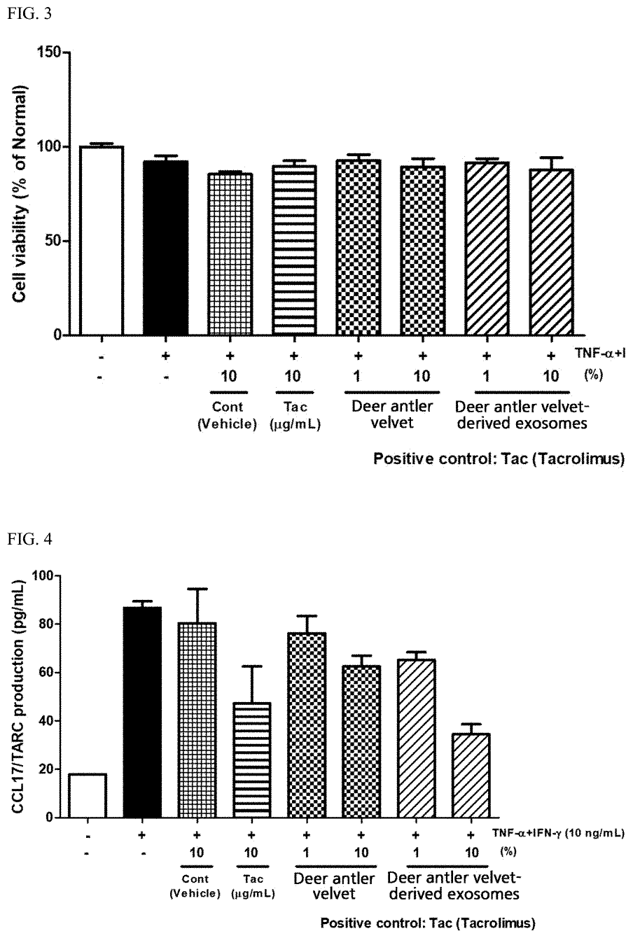

The effects of the deer antler velvet-derived exosomes prepared in Experimental Example 1 and the deer antler velvet extract prepared in Experimental Example 2 on the growth of human immortalized keratinocytes (HaCaT, ATCC) were evaluated by MTT colorimetric assay. First, human immortalized keratinocytes were inoculated into Dulbecco's modified Eagle's medium (DMEM, GIBCO) as a dedicated medium supplemented with 10% fetal bovine serum (FBS, Cambrex) in a 24-well cell culture dish at a density of 1×10⁵ and cultured under humidified conditions at 37° C. and 5% CO₂ for 24 h. After removal of the medium, the culture was treated with different concentrations (10 and 100 μg/ml) of the deer antler velvet-derived exosomes prepared in Example 1 diluted with serum-free DMEM, followed by further culture for 24 h. The resulting culture was treated with MTT reagent (1 mg/ml). 2 h later, formazan produced inside the cells as a result of the MTT treatment was dissolved using DMSO and absorbance was measured at 570 nm. For comparison, the culture was treated with tacrolimus (1 and 10 μg/ml) as a positive control. As a result, it was found that the deer antler velvet extract and the deer antler velvet-derived exosomes did not significantly affect the viability of human immortalized keratinocytes, indicating that the deer antler velvet extract and the deer antler velvet-derived exosomes did not show toxicity (FIG. 3).

### 5. Evaluation of Anti-Atopic Efficacy of the Deer Antler Velvet-Derived Exosomes

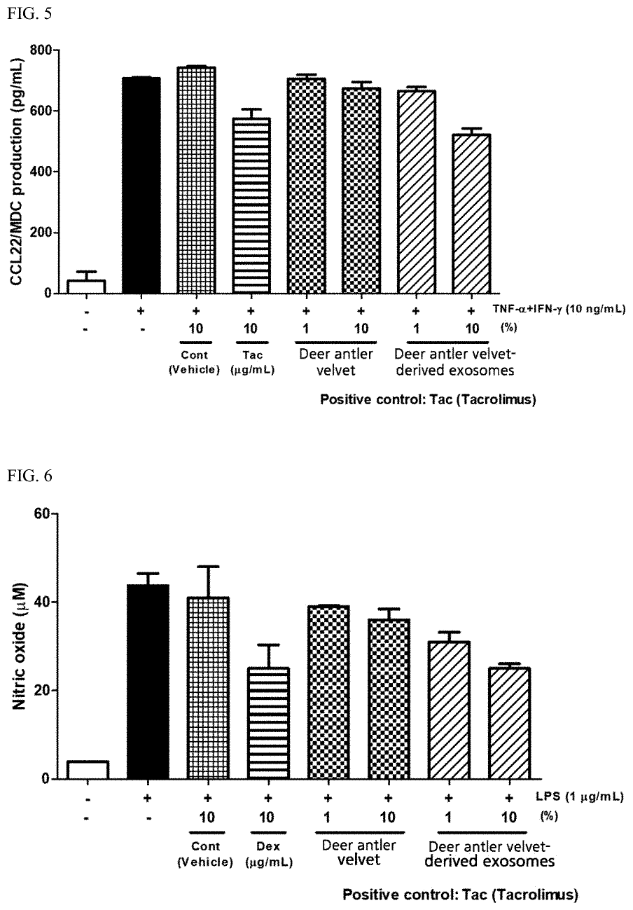

Human immortalized keratinocyte cell line (HaCaT, 5×10⁵/well) was cultured in FBS DMEM for 24 h. Cells were co-treated with different concentrations (1% and 10% (v/v)) of the deer antler velvet-derived exosomes prepared in Experimental Example 1 and TNF-α and IFN-γ (10 ng/ml) diluted with FBS-free DMEM, followed by further culture for 24 h. The above procedure was repeated except that the deer antler velvet extract prepared in Experimental Example 2 was used instead of the deer antler velvet-derived exosomes prepared in Experimental Example 1. The supernatant of each well was quantified using a human TARC ELISA kit (R&D system) and a human MDC ELISA kit (R&D system). A group treated with the same amount of distilled water was used as a negative control and a group treated with tacrolimus was used as a positive control. Tacrolimus is a calcineurin inhibitor and is known to inhibit the phosphatase activity of calcineurin (Sieber, M., Karanik, M., Brandt, C., Blex, C., et al., 2007, Inhibition of calcineurin NFAT signaling by the pyrazolopyrimidine compound NCI3., European Journal of Immunology, 37, 2617-2626). A recent research has shown that tacrolimus administration is effective in patients with atopic dermatitis resistant to corticosteroids (Russell, J. J., 2002, Topical tacrolimus: a new therapy for atopic dermatitis. American Family Physician, 66, 1899-1902). Referring to FIG. 4, the treatment with the antler velvet-derived exosomes significantly suppressed the production of overexpressed TARC induced by TNF-α and IFN-γ. This effect was significantly improved in the test group treated with the antler velvet-derived exosomes at a concentration of 10% (v/v) compared to that in the positive control treated with tacrolimus. These results indicate that the deer antler velvet-derived exosomes can inhibit the expression of thymus and activation-regulated chemokine (TARC), a specific factor for atopic dermatitis, to effectively prevent and improve atopic dermatitis. Referring to FIG. 5, the treatment with the antler velvet-derived exosomes significantly suppressed the production of overexpressed MDC induced by TNF-α and IFN-γ. This effect was significantly improved in the test group treated with the antler velvet-derived exosomes at a concentration of 10% (v/v) compared to that in the positive control treated with tacrolimus. These results indicate that the deer antler velvet-derived exosomes are effective over tacrolimus as an immunosuppressant that has been used to treat atopic dermatitis and they can be used as natural medicinal ingredients to replace tacrolimus.

### 6. Evaluation of Inhibitory Efficacy of the Deer Antler Velvet-Derived Exosomes on Nitric Oxide Production

To evaluate the anti-inflammatory effect of the deer antler velvet-derived exosomes, the inhibition of production of nitric oxide (NO), a representative cytotoxic agent involved in inflammation induction, was measured. First, RAW 264.7 cells of a macrophage cell line were obtained from the Korean Cell Line Bank (KTCC, Seoul, Korea). Specifically, the cells were inoculated into Dulbecco's modified Eagle's medium supplemented with 10% fetal bovine serum (FBS), 100 μg/ml penicillin, and 100 μg/ml streptomycin and cultured at 37° C. and 5% CO₂. More specifically, the RAW 264.7 cells were seeded in a 96-well plate at a density of 1×10⁶ cells/mL (in DMEM) and cultured for 24 h. Cells were co-treated with different concentrations (1% and 10% (v/v)) of the deer antler velvet-derived exosomes prepared in Experimental Example 1 and a fresh medium containing LPS (1 μg/ml), which is known as an endotoxin, followed by further culture for 24 h. The above procedure was repeated except that the deer antler velvet extract prepared in Experimental Example 2 was used instead of the deer antler velvet-derived exosomes prepared in Experimental Example 1. Then, 100 μl of the cell culture supernatant was mixed with 100 μl of Griess reagent [1% (w/v) sulfanilamide and 0.1% (w/v) naphthylethylenediamine in 2.5% (v/v) phosphoric acid] and incubated in a 96-well plate for 10 min. Absorbance was measured at 540 nm using an ELISA reader to determine the amount of nitric oxide produced. The concentration of nitrite produced was calculated from a standard curve of sodium nitrite in DMEM. The inhibitory activity of each sample on nitric oxide production was evaluated based on a difference in the amount of nitrite produced between the LPS-treated control (Cont) and the untreated control (Nor). As a result, the deer antler velvet-derived exosomes were observed to suppress the production of nitric oxide in all treated groups compared to in the LPS-treated control (Cont), unlike the deer antler velvet extract, indicating their excellent anti-inflammatory effect (FIG. 6).

### 7. Evaluation of Anti-Inflammatory Efficacy of the Deer Antler Velvet-Derived Exosomes

RAW 264.7 cells of a macrophage cell line were obtained from the Korean Cell Line Bank (KTCC, Seoul, Korea). Specifically, the cells were inoculated into Dulbecco's modified Eagle's medium supplemented with 10% fetal bovine serum (FBS), 100 μg/ml penicillin, and 100 μg/ml streptomycin and cultured at 37° C. and 5% CO₂. More specifically, the RAW 264.7 cells were seeded in a 6-well plate at a density of 1×10⁶ cells/mL (in DMEM) and cultured for 24 h. Cells were co-treated with different concentrations (1% and 10% (v/v)) of the deer antler velvet-derived exosomes prepared in Experimental Example 1 and a fresh medium containing LPS (1 μg/ml), which is known as an endotoxin, followed by further culture for 24 h. The above procedure was repeated except that the deer antler velvet extract prepared in Experimental Example 2 was used instead of the deer antler velvet-derived exosomes prepared in Experimental Example 1. The mRNA expression level of the COX-2 gene was determined by RT-PCR. The primers used were as follows:

COX-2:
Forward
(SEQ ID No. 1)
5′-AAG ACT TGC CAG GCT GAA CT-3′,
Reverse
(SEQ ID No. 2)
5′-CTT CTG CAG TCC AGG TTC AA-3′.
GAPDH:
Forward
(SEQ ID No. 3)
5′-TGA AGG TCG GTG TGA ACG GAT TTT GGC-3′,
Reverse
(SEQ ID No. 4)
5′-TGG TTC ACA CCC ATC ACA AAC ATG G-3′.

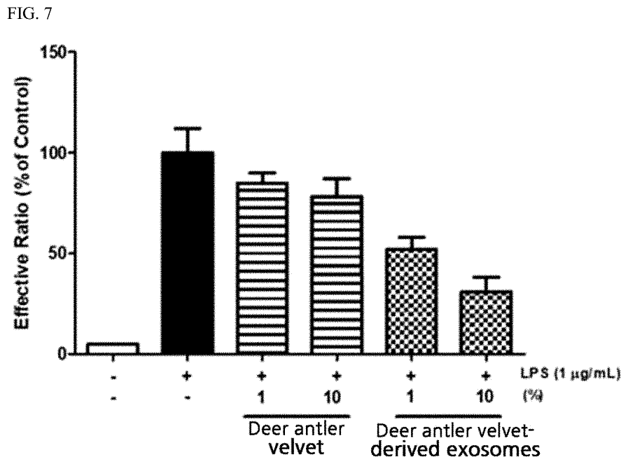

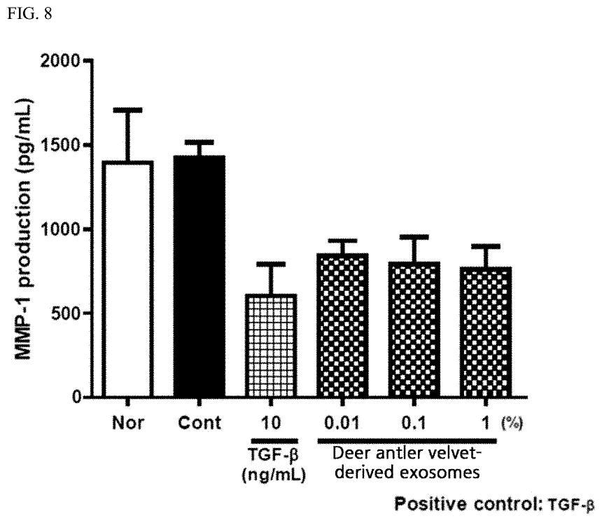

RT-PCR was performed using the above primers. The experimental results are summarized in FIG. 7. A significant inhibitory effect on COX-2 gene expression was observed in the group treated with the deer antler velvet-derived exosomes compared to in the negative control treated with LPS only and the group treated with the deer antler velvet extract, indicating that the deer antler velvet-derived exosomes can be used to treat and prevent inflammatory diseases or are effective in skin soothing.

### 8. Evaluation of Anti-Wrinkle Effect of the Deer Antler Velvet-Derived Exosomes

Normal human dermal fibroblasts (NHDFs) were obtained by skin biopsy from healthy young male volunteers. Cells were plated in a 100 mm tissue culture plate and cultured in DMEM supplemented with 10% heat-inactivated FBS and 1% penicillin-streptomycin in a humid environment of 5% CO₂ at 37° C. All experiments were conducted using only cells between passages 6 and 10. The normal human dermal fibroblasts were seeded in 40-mm tissue culture plates (1.2×10⁵ cells), cultured to a confluence of 80%, and washed twice with phosphate buffered saline (PBS), after which 1980 μl of fresh serum-free medium and 20 μl of sample were added to each well. After culture for 3 days, the culture was harvested and centrifuged at 7,500 rpm and 4° C. for 5 min. A change in the expression level of MMP-1 protein (Human Total MMP-1 kit (R&D Systems, Inc., Minneapolis, Minn., USA)) was monitored by ELISA. The treatment with the deer antler velvet-derived exosomes prepared in Experimental Example 1 led to a marked reduction in the expression of MMP-1 protein. Specifically, the inhibitory effects of the groups treated with the deer antler velvet-derived exosomes at concentration of 0.01%, 0.1%, and 1% were improved by 41%, 45%, and 47%, respectively, compared to those of the untreated groups (FIG. 8). These results indicate that the deer antler velvet-derived exosomes can effectively suppress the production of MMP-1 protein, which is the cause of wrinkles, to prevent and ameliorate skin wrinkles.

### PREPARATIVE EXAMPLES

### Preparative Example 1: Preparation of Cosmetic Product

According to a suitable process known in the art, a cosmetic product was produced to have the following composition: 2 ml of the deer antler velvet-derived exosomes, 7.0 mg of 1,3-butylene glycol, 1.0 mg of glycerin, 1.5 mg of polysorbate 60, 2.0 mg of lipophilic glyceryl stearate, 4.0 mg of mineral oil, 3.0 mg of cetearyl alcohol, a small amount of a preservative, an appropriate amount of a combined fragrance, and purified water (up to a total of 100 mg).

### Preparative Example 2: Preparation of Pharmaceutical Products

A 0.9% isotonic solution (pH 7.0) was prepared using 2 ml of the deer antler velvet-derived exosomes, additives such as NaCl and NaOH, and 900 ml of water for injection. The isotonic solution was filtered three times through a PES 0.1 filter. After glass vial bottles, rubber stoppers, and aluminum caps were sterilized, the isotonic solution was divided into small portions (each 20 ml) and placed in the vial bottles. The vial bottles were capped with the rubber stoppers and the aluminum caps and sterilized in an autoclave under high pressure. After foreign matter was inspected using a foreign matter inspection machine, microbiological and endotoxin tests were conducted. Thereafter, the vials were labeled and kept refrigerated.

### Preparative Example 3: Preparation of Granules

According to a suitable process known in the art, 2 ml of the deer antler velvet-derived exosomes, an appropriate amount of a vitamin mixture, 10 μg of biotin, 1.7 mg of nicotinamide, 50 μg of folic acid, 0.5 mg of calcium pantothenate, an appropriate amount of a mineral mixture, 1.75 mg of ferrous sulfate, 0.82 mg of zinc oxide, 25.3 mg of magnesium carbonate, 15 mg of potassium phosphate monobasic, 55 mg of dibasic calcium phosphate, 90 mg of potassium citrate, 100 mg of calcium carbonate, and 24.8 mg of magnesium chloride were mixed to prepare a health functional food.

### Preparative Example 4: Preparation of Mask Packs

According to a suitable process known in the art, 0.05 wt % of the deer antler velvet-derived exosomes, 5.0 wt % of glycerin, 4.0 wt % of propylene glycol, 15.0 wt % of polyvinyl alcohol, 8.0 wt % of ethanol, 1.0 wt % of polyoxyethylene ether, 0.2 wt % of methyl para-oxybenzoate, an appropriate amount of a dye, and an appropriate amount of a fragrance were mixed to prepare mask packs.

Although the particulars of the present invention have been described in detail, it will be obvious to those skilled in the art that such particulars are merely preferred embodiments and are not intended to limit the scope of the present invention. Therefore, the substantial scope of the present invention is defined by the appended claims and their equivalents.

What is claimed is:

1. A method for soothing skin in a subject in need thereof, comprising administering to the subject a composition comprising deer antler velvet-derived exosomes as active ingredients.

2. The method according to claim 1, wherein the skin soothing is anti-inflammation, amelioration of atopic dermatitis, antioxidation or wrinkle improvement.

3. The method according to claim 1, wherein the exosomes have a diameter of 120 to 600 nm.

4. The method according to claim 1, wherein the composition is prepared into a powder, gel, cream, essence, lotion, sol-gel, emulsion, oil, wax, spray or mist.

5. The method according to claim 1, wherein the composition is prepared into a mask pack.

6. A method for ameliorating or treating dermatitis, atopic dermatitis or wrinkle in a subject in need thereof, comprising administering to the subject a composition comprising deer antler velvet-derived exosomes as active ingredients.

7. A method for preparing a skin soothing composition comprising deer antler velvet-derived exosomes as active ingredients, the method comprising a) washing and powdering deer antler velvet, b) dissolving the powder in an exosome extraction buffer, and c) isolating deer antler velvet-derived exosomes from the solution.
